(19) United States
(12) Patent Application Publication
(10) Pub. No.: US 2024/0165410 A1
(43) Pub. Date: May 23, 2024

(54) MODULATING WAKEFULNESS AND AROUSAL

(71) Applicant: Oxford University Innovation Limited, Oxford (GB)

(72) Inventors: Alkistis Stavropoulou DELI, Oxford (GB); Timothy DENISON, Oxford (GB); Alexander Laurence GREEN, Oxford (GB)

(21) Appl. No.: 18/282,561

(22) PCT Filed: Mar. 28, 2022
(86) PCT No.: PCT/GB2022/050763

§ 371 Date: Sep. 18, 2023

(30) Foreign Application Priority Data
Mar. 26, 2021 (GB) 2104315.3

(51) Int. Cl. A61N 1/36; A61M 21/00

(52) U.S. Cl. CPC A61N 1/36078; A61M 21/00; A61N 1/36139; A61N 1/36171; A61M 2021/0072; A61M 2021/0083; A61M 2230/10; A61M 2230/18

(57) ABSTRACT

Arousal of a patient is enhanced and wakefulness modulated by neurostimulation. Bioelectrical activity of the nervous system of the patient is monitored and characteristics of the monitored bioelectrical activity associated with a state of reduced arousal and/or wakefulness are detected. In response to such characteristics, stimulation signals selected to arouse the patient are generated and supplied to stimulation transducers to stimulate a neural network of a patient associated with arousal.

The present invention relates to the use of stimulation to modulate arousal and/or wakefulness of a patient.

There are a number of disorders and applications where excessive sleepiness can have severe implications. For example, hypersomnia is a common and debilitating condition of bidirectional interactions with mental health conditions such as low mood (Dauvilliers et al. 2013) that can further impact quality of life and daily functioning. There are no FDA-approved treatments for idiopathic hypersomnia, with patients relying on off-label stimulants with variable results and risks (Saini and Rye, 2017). Further down the end of the clinical spectrum, patients with narcolepsy suffer from daytime sleep attacks, the consequence of a difficulty in maintaining vigilance states (Schoch et al. 2017) secondary to loss of orexin-secreting neurons in the hypothalamus.

Dysregulated sleep/decreased arousal is also a common feature of neurodegenerative disorders (reviewed in Bhat and Chokroverty 2017). Consequently, there exists a population of patients with deep brain implants for neurodegenerative movement disorders who suffer from excessive drowsiness and/or pathological sleep. Some brain areas implanted for current clinical indications for deep brain stimulation (DBS) (including thalamo-cortical, basal ganglia, and brain stem regions) may show a degree of overlap with circuits involved in sleep maintenance and regulation (e.g. Sharma et al 2018). However, there exists no targeted DBS therapy for sleep disorders.

Decreased arousal during the day and increases in arousal at night are also seen in disorders of consciousness, such as vegetative and minimally conscious state. This disruption is a consequence of impaired circadian rhythms, whose level of impairment correlates to disease severity (vegetative patients were significantly more impaired than minimally conscious patients, Cruse et al. 2013). In addition, several studies have shown that brain activity patterns associated with drowsiness and sleep (such as slow wave activity) are increasingly present in patients with a disorder of consciousness outside of sleep or sleep-like behaviours (at resting-state, for example Sitt et al. 2014).

In addition to disorders involving excessive drowsiness and prolonged/mistimed sleep, there are high-performance occupations where loss of vigilance and episodes of micro-sleep can have fatal consequences. Measures monitoring vigilance in professionals such as pilots were of early interest (e.g. Samn Perelli 1982), but there is no naturalistic, non-pharmacological way to maintain wakefulness while monitoring its decline in real time.

As such, it would be desirable to provide ways of maintaining wakefulness.

According to a first aspect of the invention there is provided a method of generating stimulation signals for modulating the arousal and/or wakefulness of a patient, the method comprising: monitoring bioelectrical activity of the nervous system of the patient; detecting characteristics of the monitored bioelectrical activity associated with a state of reduced arousal and/or wakefulness; and in response to detecting said characteristics of the monitored bioelectrical activity associated with a state of reduced arousal and/or wakefulness, generating stimulation signals for supply to transducers arranged to apply electromagnetic stimulation to a neural network of a patient associated with arousal, the stimulation signals being selected to arouse the patient when the stimulation is applied.

Application of such a method in a stimulation device provides for modulation of arousal levels of a patient, and thus helps maintain wakefulness of the patient by the subsequent supply of the stimulation signals to the neural network of the patient. This provides for a naturalistic, non-pharmacological way to maintain wakefulness while monitoring its decline in real time.

In some embodiments, the detected characteristics are indicative of a stage of sleep, for example a pre-sleep stage, non-REM Sleep Stage 1, non-REM Sleep Stage 2, non-REM Sleep Stage 3, or REM sleep. The pre-sleep stage may comprise a period of reduced wakefulness and/or arousal prior to actual sleep. By detecting the sleep stage, appropriate stimulation for that stage may be applied.

In some embodiments, the detected characteristics comprise an indication of sleep fragility in the patient. The stimulation signals are synchronized (e.g. timed to generate stimulation at the same time as or during occurrence of the indication of sleep fragility; or within a fixed time of the indication of sleep fragility) with the indication of sleep fragility. By matching the stimulation to natural moments of sleep fragility, the impact of the stimulation of the level of arousal in the patient may be increased. Detected characteristics which are indicative of sleep fragility may also be indicative of a sleep stage. Alternatively, the bioelectrical activity may comprise both characteristics indicative of a sleep stage and distinct characteristics indicative of sleep fragility (of which the former can be used to identify the reduced wakefulness and/or arousal; and the latter can be used to synchronize the stimulation).

In some embodiments, the method further comprises receiving a signal indicative of peripheral activity of the patient; and detecting characteristics in the signal indicative of peripheral activity associated with a reduced state of wakefulness. The stimulation signal is then generated in response to detecting the characteristics of the monitored bioelectrical activity and the characteristics in the signal indicative of peripheral activity. This may apply to stimulation generated both in response to bioelectrical characteristics indicative of a sleep stage, and in response to bioelectrical characteristics indicative of sleep fragility. Such embodiments allow peripheral (i.e. non-neurological or non-central neurological) activity of the patient, such as physical or cardiovascular activity, to be taken into account when identifying that the patient is in a state of reduced arousal/wakefulness. This may provide for more accurate and timely recognition of the reduced state of arousal and/or wakefulness.

In some such embodiments, the indication of sleep fragility may also be used to detect the state of reduced arousal or wakefulness.

According to a further aspect of the present invention, there is provided a stimulation device that implements a similar method to the first aspect of the present invention. In particular, there is provided a stimulation device for generating stimulation signals for modulating the wakefulness and/or arousal level of a patient, the stimulation device comprising: a detection unit arranged to monitor bioelectrical activity of the nervous system of the patient and to detect characteristics of the monitored bioelectrical activity associated with a state of reduced arousal and/or wakefulness; and a stimulation generator arranged, responsive to detecting said characteristics of the monitored bioelectrical activity associated with a state of reduced arousal and/or wakefulness, to generate stimulation signals for supply to transducers arranged to apply electromagnetic stimulation to a neural network of a patient associated with arousal, the stimulation signals being selected to arouse the patient when the electrical stimulation is applied.

In some embodiments the detection unit may comprise a sleep stage detector arranged to detect characteristics indicative of a state of sleep of the patient in the monitored bioelectrical activity.

In some embodiments, the detection unit may comprise a sleep fragility detector arranged to detect an indication of sleep fragility in the patient in the monitored bioelectrical activity; wherein the stimulation generator is configured to synchronise the stimulation signal with the detected indication of sleep fragility.

In some embodiments the stimulation device may further comprise an interface unit configured to receive signals indicative of peripheral activity of the patient.

To allow better understanding, embodiments of the present invention will now be described by way of non-limitative example with reference to the accompanying drawings, in which:

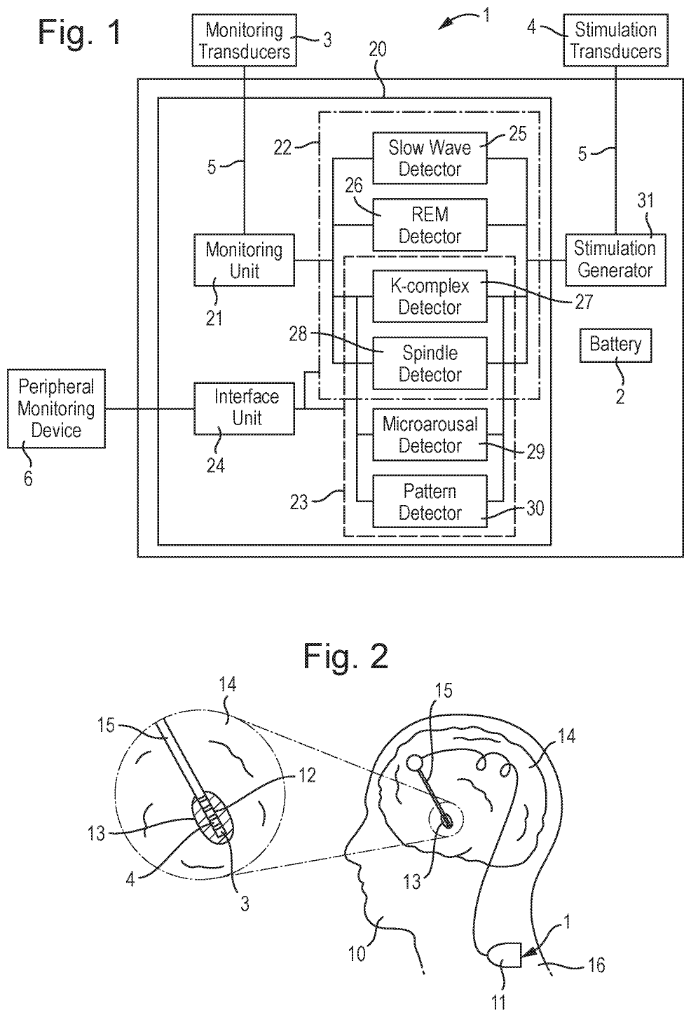

FIG. 1 is a schematic diagram of a stimulation device;

FIG. 2 is a diagram of a stimulation device with implanted electrodes;

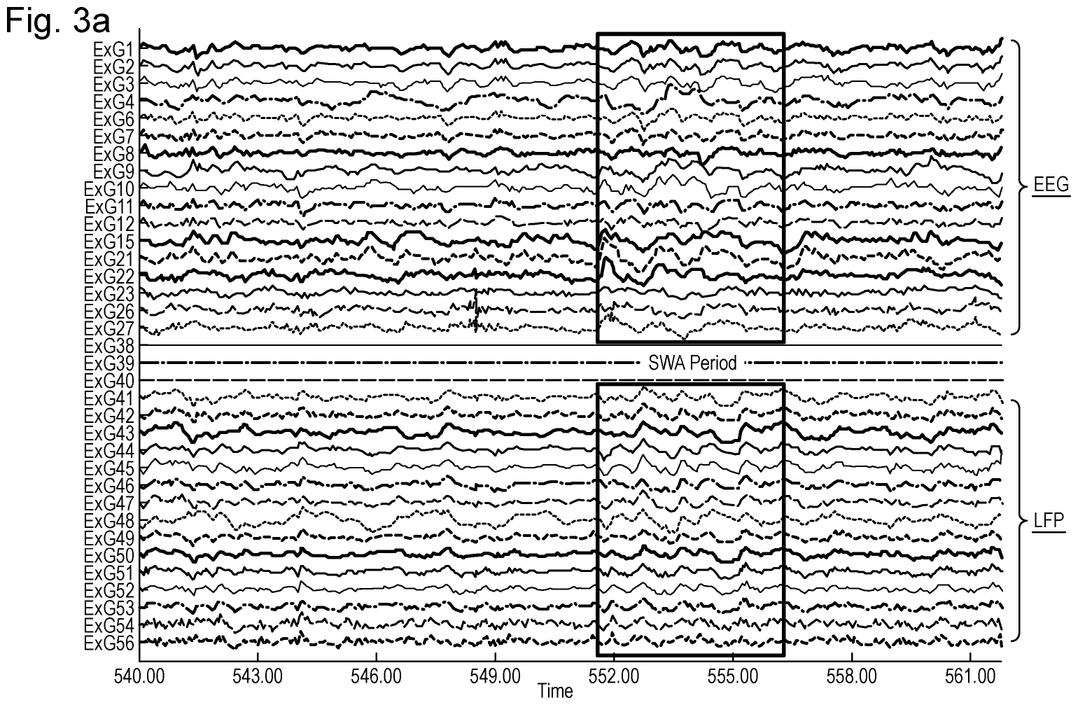

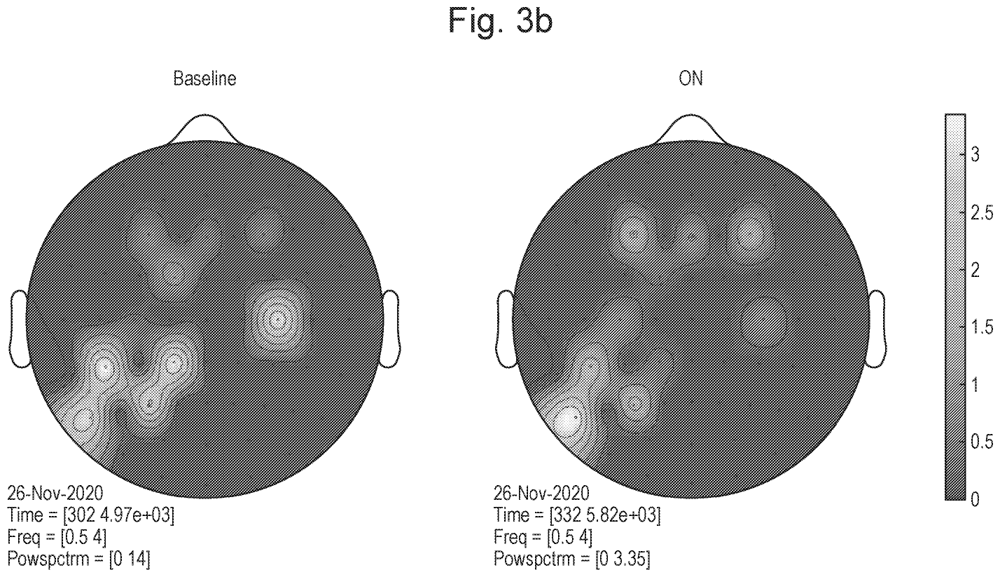

FIG. 3a is a plot of the monitored surface (EEG) signal as well as the signal from a deep brain area (LFP) over time, showing the presence of slow wave activity (SWA);

FIG. 3b is a series of plots from a case study, showing how slow wave activity decreases when deep brain stimulation is delivered

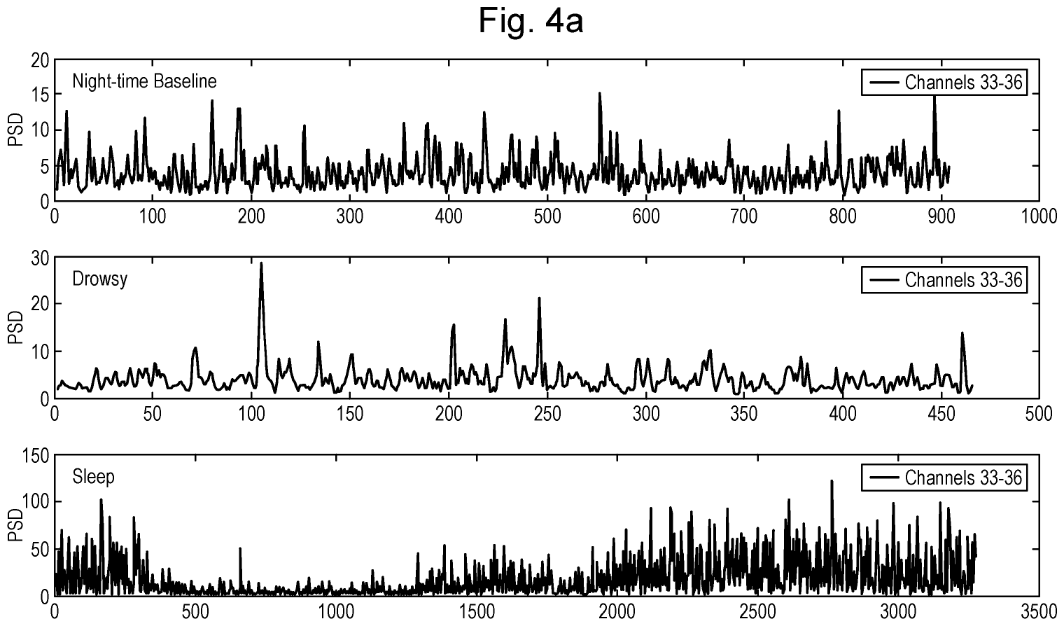

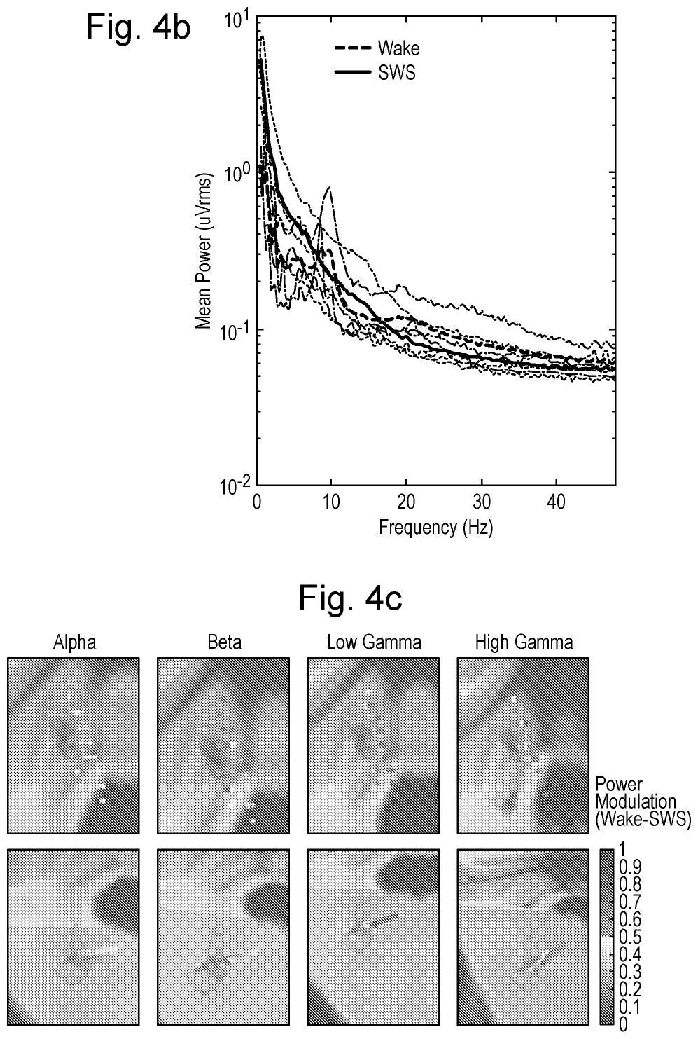

FIG. 4a is a plot of signals from a deep brain electrode (located in the insular cortex) showing an increase in slow wave activity as the level of arousal decreases;

FIG. 4b is a plot of the signals from a deep brain electrode (located in the pedunculopontine nucleus of the brainstem) showing an decrease in beta and gamma activity as the level of arousal decreases;

FIG. 4c is a set of images of the mapped space corresponding to the measurements of FIG. 4b;

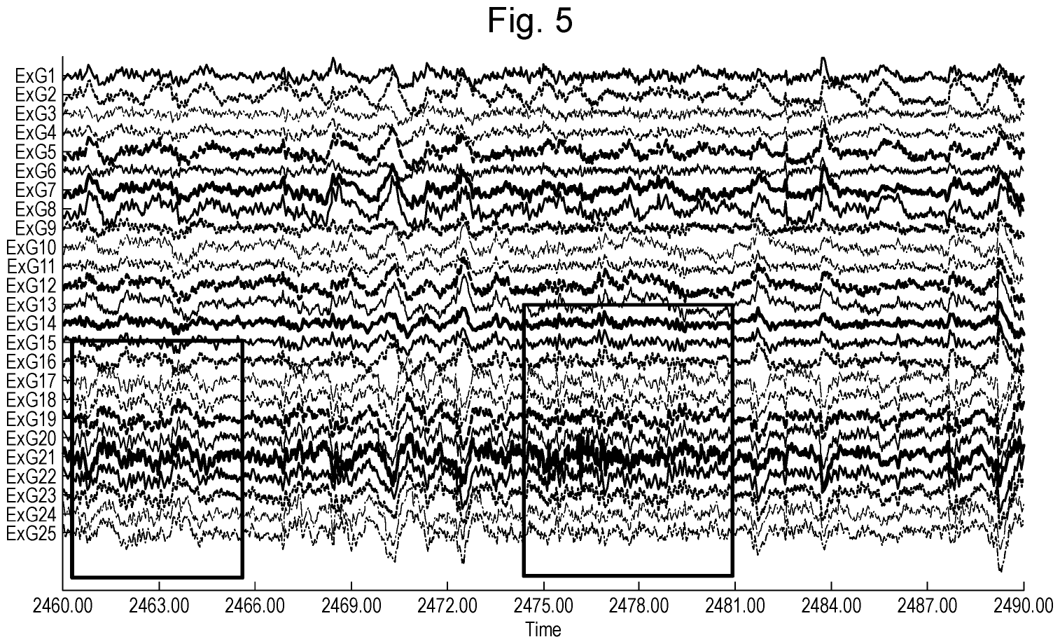

FIG. 5 is a plot of the monitored surface (EEG) signal over time, showing the presence of spindles;

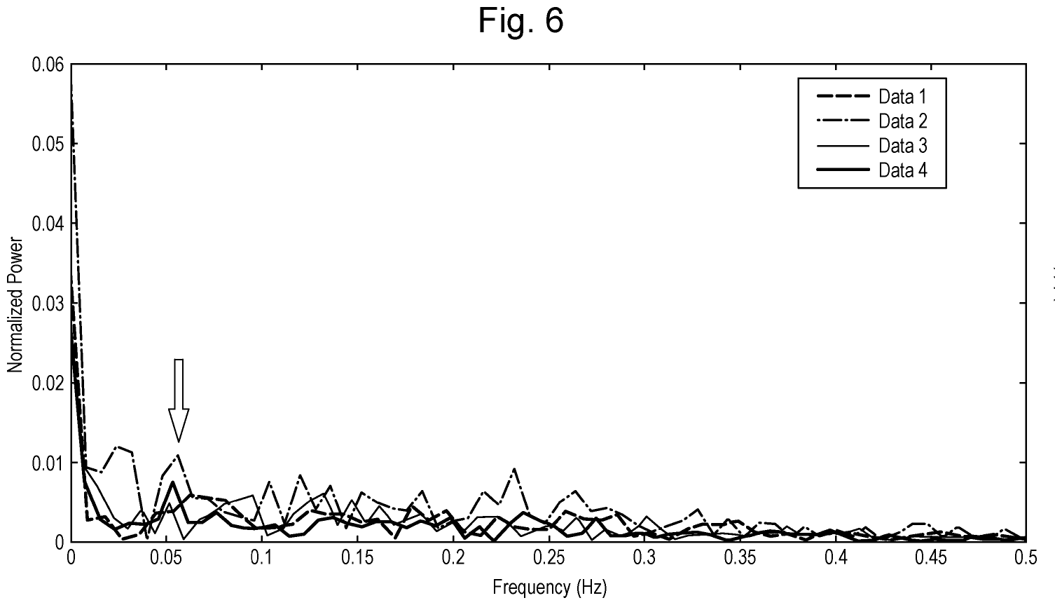

FIG. 6 is a graph of normalised PSD against frequency in an experimental study, illustrating the presence of peaks in the periodicity of sleep spindle power;

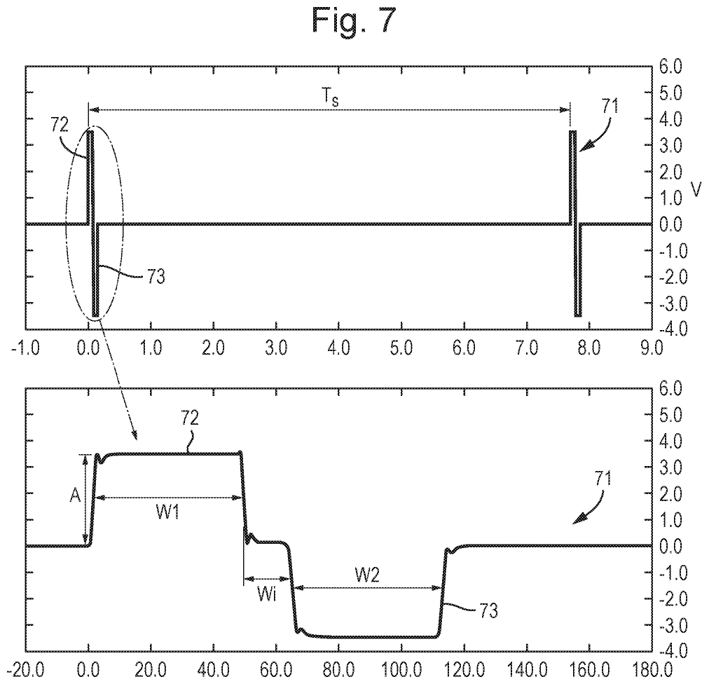

FIG. 7 is a pair of graphs illustrating a typical stimulation signal;

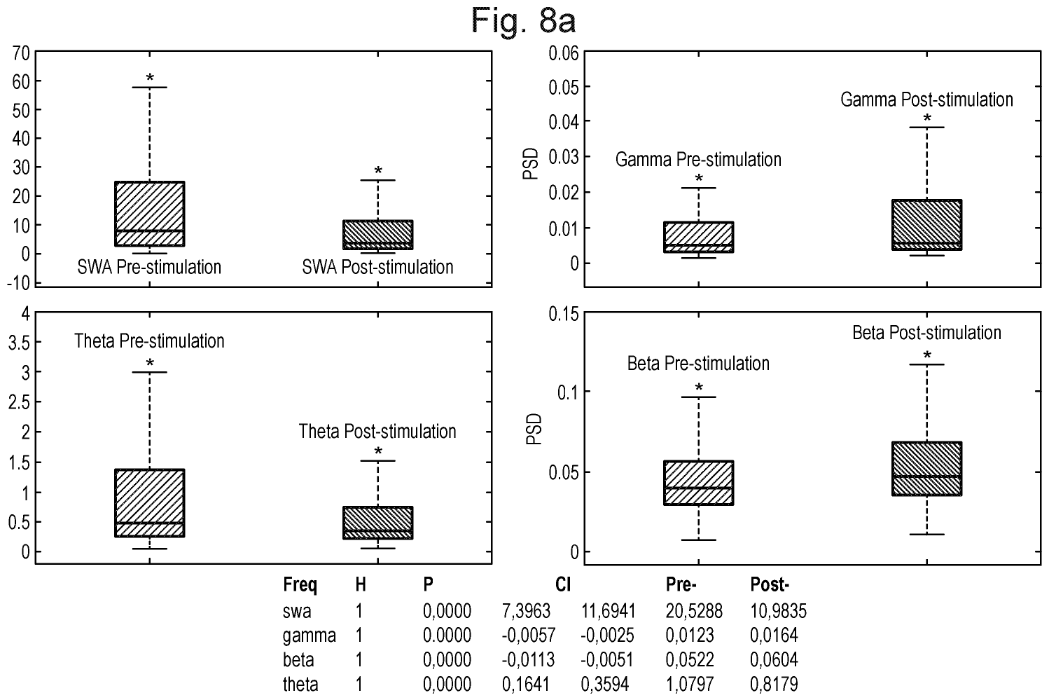

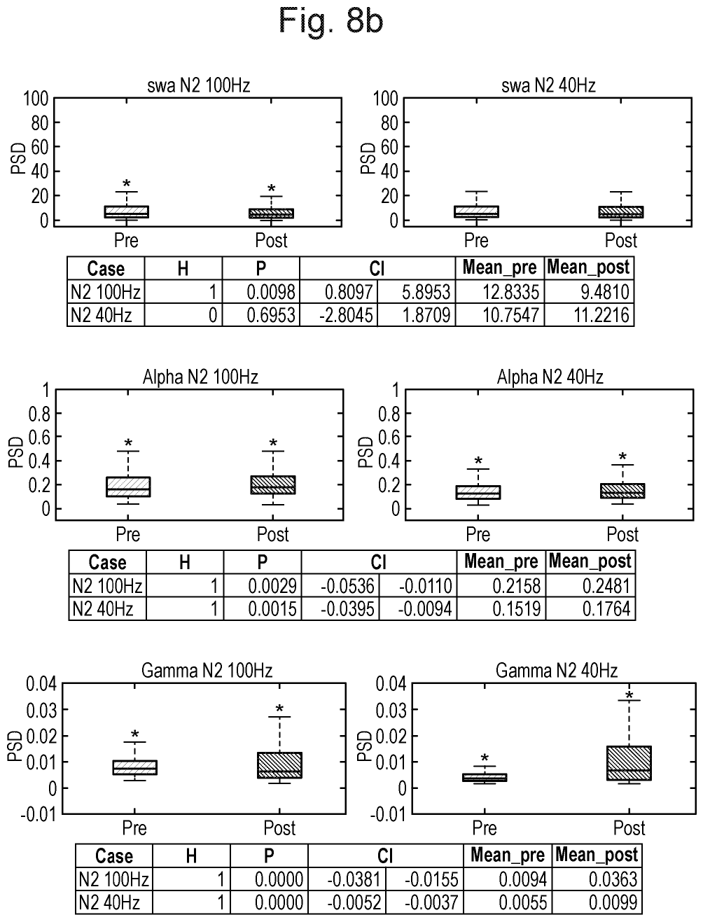

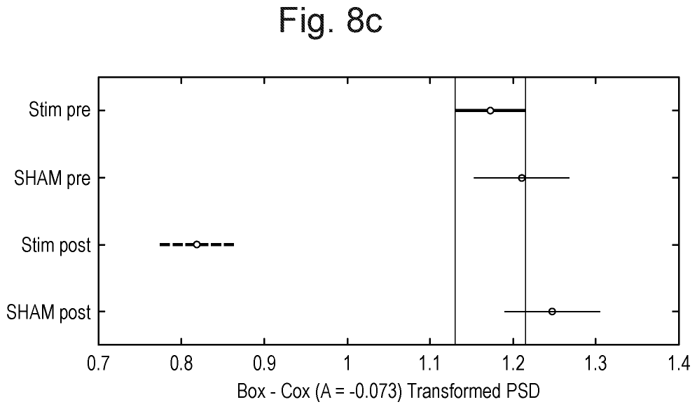

FIG. 8a is four graphs and a table showing results for a study performing bilateral stimulation of a patient demonstrating enhancement of rhythms associated with increased arousal and decrease of rhythms associated with sleep/low arousal levels;

FIG. 8b illustrates results of a study of performing unilateral stimulation to study the efficacy in reducing SWA and increasing rhythms associated with wakefulness/lighter sleep;

FIG. 8c illustrates results of performing stimulation of brainstem circuits (unilateral and bilateral) in reducing SWA, showing the efficacy of stimulation compared to sham trials where no stimulation was delivered;

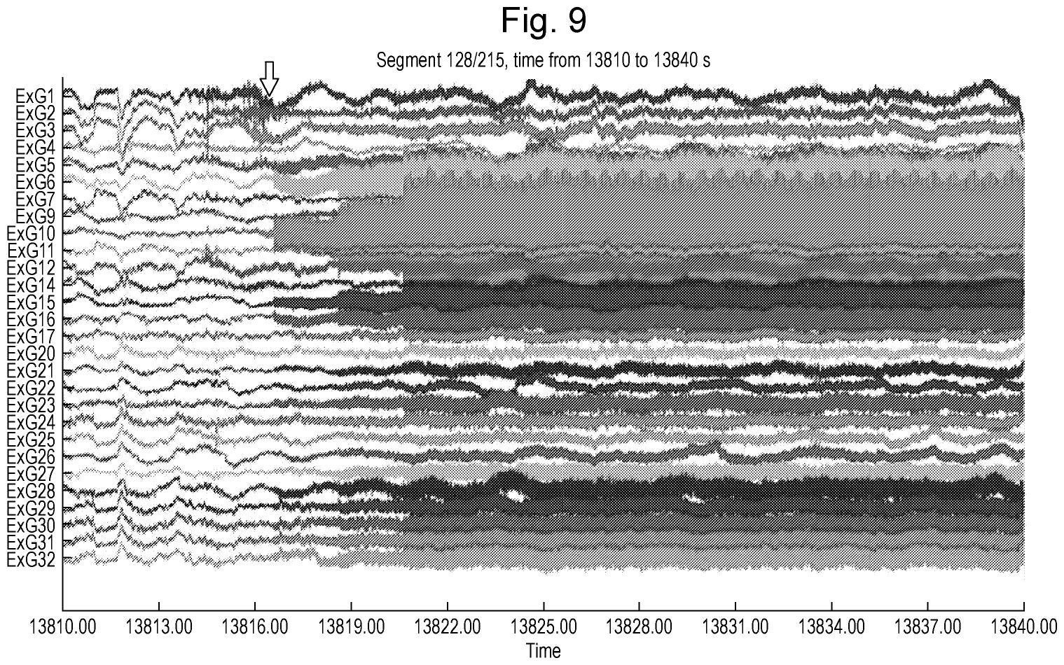

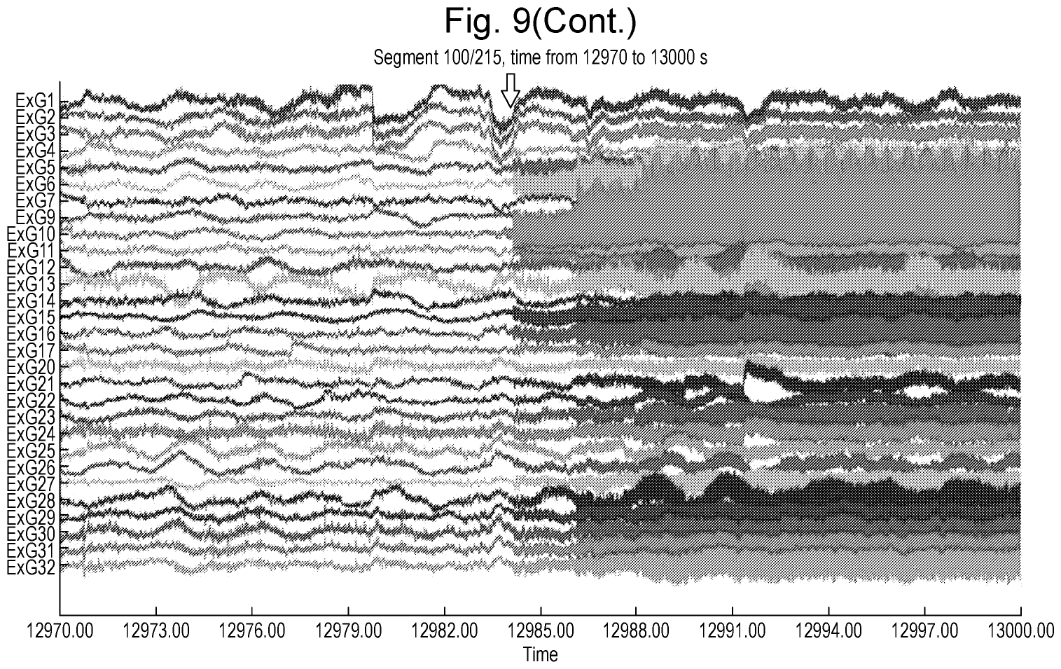

FIG. 9 shows a set of bioelectrical signals from EEG monitoring electrodes during the delivery of stimulation signals in response to detection of k-complexes;

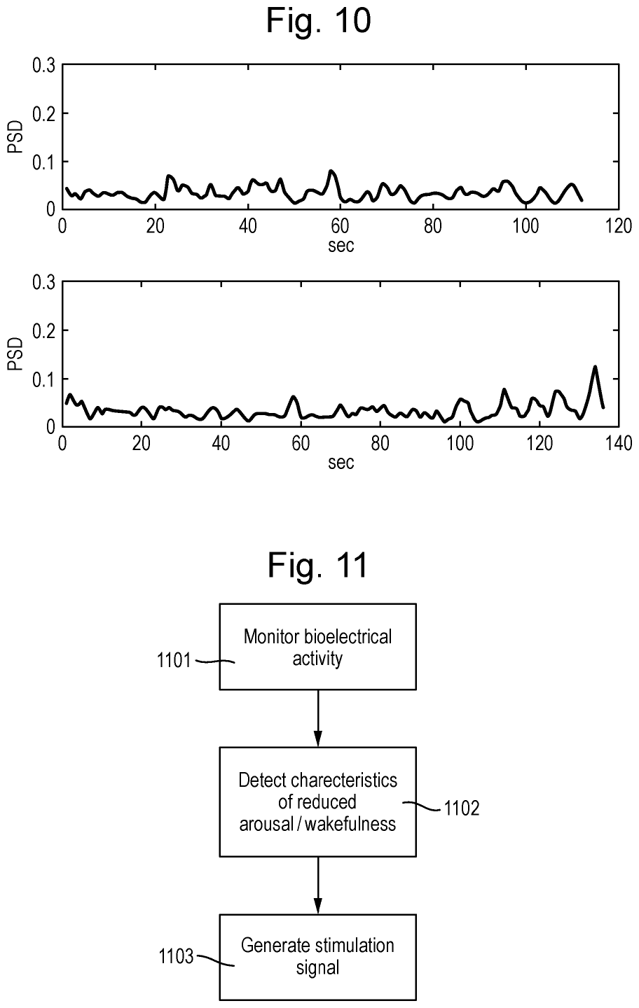

FIG. 10 is a set of graphs of PSD of sleep spindles over time pre-stimulation in trials where stimulation signals were applied in association with a decrease in sleep spindle power; and

FIG. 11 illustrates a method of modulating wakefulness and/or arousal levels of a patient.

FIG. 1 shows a schematic of a stimulation device 1 which implements a method of generating stimulation signals for enhancing the arousal of a patient. The stimulation device 1 may be worn or carried by the patient, or may alternatively be implanted into the body of the patient. The patient may be a human or animal patient. The stimulation device 1 is powered by a battery 2.

The stimulation device 1 is connected to monitoring transducers 3 and stimulation transducers 4 by a wires 5. The monitoring and/or stimulation transducers may for example be electrodes or coils (or any other electromagnet). In various embodiments, the monitoring transducers 3 and stimulation transducers 4 may or may not be considered part of the stimulation device 1. The wires 5 may be connected to the stimulation device 1 detachably or permanently attached to the stimulation device 1.

The stimulation transducers 4 are used to apply electromagnetic stimulation to a target region of the patient. The electromagnetic stimulation may be electric or magnetic stimulation, or both. Stimulation signals generated by a stimulation generator 31 described further below are supplied to the stimulation transducers 4 to apply the stimulation. Where stimulation transducers 4 are or comprise electrodes, the stimulation signal may cause an electrical stimulation to be applied to the target region. Where the stimulation transducers 4 are or comprise coils, the stimulation signal may cause a magnetic or both electric and magnetic stimulation to the target region.

The target region may in general be any neural network of the patient associated with arousal. In order to most effectively modulate alertness and sleep depth, the target region desirably has a primary physiological role in the regulation of these functions. By way of example, an area with such a role, located in the basal forebrain, was first described in von Economo (1917) which is considered classic work on the neuroanatomy of arousal circuits. Selective stimulation of neuronal populations in regions such as the locus coeruleus or the lateral hypothalamus using optogenetic techniques have successfully induced wakefulness in animals (e.g. in Carter et al 2010, Adamantidis et al 2007).

Typically, the target region may be part of the ascending arousal system (also known as reticular activating system or RAS). In a very simple schematic description, the ascending arousal system has two major branches (as disclosed in Saper et al. 2005). First, a pathway starting at the upper brainstem (regions such as the pedunculopontine nucleus (PPN) and the laterodorsal tegmentum (LDT)) reaches thalamic nuclei (relay nuclei and the reticular nucleus), delivering activating projections to the cerebral cortex. A second branch originating from the locus coeruleus and dorsal raphe, bypasses the thalamus and through hypothalamic and basal forebrain areas projects to cortex. Although there is interplay between these two branches, this theory may imply that we can still ‘rescue’ arousal in cases where the hypothalamus is destroyed by autoimmune processes, through stimulation of main nuclei of the other branch. The target region may be in either of these branches, for example in the PPN or LDT.

Optionally, stimulation transducers 4 may be provided in more than one target region that is a neural network of the patient associated with arousal, for example in each of the branches of the ascending arousal system. Such a multi-target approach may be advantageous for severe cases of decreased arousal levels and/or where more than one branch have been impacted by disease. In addition, stimulation transducers 4 may be combined with a ‘traditional’ lead or target for motor symptom control. Such an approach may be suitable for example when hypersomnia is a debilitating symptom of another primary indication for DBS.

The stimulation transducers 4 may be implanted in the target region. In this case, the stimulation is deep brain stimulation (which may be electrical, magnetic, or both, as discussed above).

FIG. 2 illustrates an example of a stimulation device 1 having stimulation transducers 4 implanted in a patient 10, which is configured as follows. The stimulation device 1 comprises a housing 11 that houses the electronic components (described in detail below). The housing 11 is connected by the lead wire 15 that includes the wires 5 providing parallel electrical connection to stimulation transducers 4 and monitoring transducers 3 that are formed on the tip 12 of the lead wire 15. In general, any number of stimulation transducers 4 and monitoring transducers 3 may be provided, the number shown in FIG. 2 being a non-limitative example. Similarly, the monitoring transducers 3 may be provided elsewhere, as discussed below.

The stimulation device 1 is surgically implanted by the tip 12 of the lead wire 15 being implanted into a target region 13 in the brain 14 of the patient 10 through a small opening in the skull of the patient 10. The housing 11 is implanted into the thorax 16 of the patient 10 near the collarbone, with the lead wire 15 extending under the skin of the patient 10. The stimulation device 1 is provided with a ground that is normally placed far away from the electrodes 5. When the overall deep brain stimulation (DBS) is internalised, the ground 10 may be connected to the housing of the neurostimulator 11 implanted under the skin at the thoracic level, although during externalization, e.g. for research purpose, the ground 10 may be taken from a part of the skin that minimises electrocardiogram (ECG) artefacts, such as the arm, neck or shoulder.

The use of implanted stimulation transducers 4 is likely the most effective option because it causes the stimulation directly in the target region. However, this option requires implantation of the stimulation transducers 4. Such a surgical procedure carries some risk. However, this option may be suitable in severe cases of, for example, treatment-resistant narcolepsy or disorders of consciousness, as well as in cases where electrodes are implanted for deep brain stimulation to treat another condition, such as Parkinson's disease, dystonia, essential tremor, or epilepsy (where multiple electrodes may also be implanted).

Alternatively, the stimulation transducers 4 (and/or monitoring transducers 3, as discussed below) may be outside the target region. For example, the stimulation transducers 4 and may be implanted subdurally or held externally of the scalp of the patient. The stimulation transducers 4 may be positioned externally or subdurally such that they are in electromagnetic communication with the target regions of the brain discussed above. In this case, the stimulation may be transcranial electrical stimulation or magnetic stimulation or both electrical and magnetic stimulation. This option may be useful for patients where excessive daytime sleepiness or hypersomnia is an issue regardless of condition, as an adjunct to stimulant pharmacotherapy or to enhance and maintain wakefulness on its own.

The monitoring transducers 3 are used to monitor bioelectrical activity of the nervous system of the patient. Various configurations for the monitoring transducers 3 are possible to monitor different types of bioelectrical activity from which reduced wakefulness may be detected. The monitoring transducers 3 may be bundled with stimulation transducers 4 in a common transducer pad. In some examples, transducers may be operable as both stimulation transducers 4 and monitoring transducers 3.

One option is to monitor biological activity derived from monitoring transducers 3 that may be implanted in the neural network of the patient that is the target region. This option may typically be applied in the case that the stimulation transducers 4 are also implanted in the target region. In such a case, the monitoring transducers 3 may also be integrated with the stimulation transducers 4, as discussed above in relation to FIG. 2. In this case, the bioelectrical activity which is monitored is deep brain activity. Such deep brain electrodes may be termed local field potential (LFP) electrodes.

Another option is to monitor biological activity by electroencephalography (EEG). In this case, the monitoring transducers 3 may be EEG electrodes implanted subdurally or placed externally on the scalp of the patient. In this case, the bioelectrical activity which is monitored is cortical activity. This option may typically be applied in the case that stimulation transducers 4 are not implanted in the target region, but could also be applied in the case that stimulation transducers 4 are implanted in the target region.

The monitoring transducers 3 may include electrodes of different types, for example both EEG electrodes and electrodes implanted in the target region.

The electronic components of the stimulation device 1 will now be described.

The stimulation device 1 comprises a detection unit 20 which is supplied with the signal from the monitoring transducers 3 and uses that signal to monitor the bioelectrical activity of the patient and detect characteristics of the monitored bioelectrical activity associated with a state of a reduced level of arousal or wakefulness, as follows.

The detection unit 20 comprises a monitoring unit 21 which receives the signal from the monitoring transducers 3. The monitoring unit 21 amplifies and filters the signal from the monitoring transducers 3 to derive bioelectrical signals representing the bioelectrical activity. This may apply to cortical or deep brain contacts—in the case of deep brain electrodes, these contain a number of points that essentially function as signal-collector contacts in a similar fashion to surface EEG electrodes. FIG. 3a shows an example of signals collected from multiple points, with a region of slow wave activity (SWA) indicated. The signals from multiple points may be averaged or otherwise combined to generate a common bioelectrical signal, or the separate signals may passed to the sleep stage detector 22 or sleep fragility detector 23 discussed below. Signals will be filtered and amplified in similar ways to remove noise and artefacts and enable detection of the characteristics described below.

In the illustrated device 1, the derived bioelectrical signals are passed to sleep stage detector 22. Sleep stage detector 22 detects characteristics of the monitored bioelectrical activity indicative of a stage of sleep of the patient. For example, the sleep stage detector 22 may detect that the patient is in a pre-sleep stage, non-REM sleep stage 1, non-REM sleep stage 2, non-REM sleep stage 3, or REM sleep. The stimulation may be triggered in response to the detection of the sleep stage.

The illustrated device 1 also comprises a sleep fragility detector 23. Sleep fragility detector is arranged to detect indications of sleep fragility (i.e. moments of increased susceptibility to stimulation) in the bioelectrical activity of the patient. The stimulation can then be synchronized with the indications of sleep fragility, to optimise the timing of the stimulation.

Providing both the sleep state detector 22 and the sleep fragility detector 23 may allow sleep fragility to be detected using features which on their own may not be sufficient to determine that the patient is in a sleep or pre-sleep stage. It is to be noted, however, that other embodiments may omit either the sleep stage detector 22 or the sleep fragility detector 23. In particular, where an indication of sleep fragility may itself be an indication that the patient is in a reduced state of arousal and/or wakefulness, and so there may be no need for a separate sleep stage detector 22.

The bioelectrical signals are supplied to the sleep stage detector 22 to detect characteristics associated with a patient's sleep (or pre-sleep) stage. As discussed further below, the detection may make use of peripheral activity of the patient, such as physical activity measured by one or more peripheral monitoring devices 6 (which may or may not be part of the device 1). Signals indicative of this peripheral activity may be provided to the sleep stage detector 22 via an interface unit 24, which in turn receives signals from the one or more peripheral monitoring devices 6. A peripheral monitoring devices 6 may for example be an actigraph, or autonomic monitoring device. Such peripheral activity information may contain information relevant to decreases in activity, so the combination to peripheral activity and bioelectrical activity may be used to predict a change in arousal/wakefulness. In a particular example, this combination may be used to predict a transition from wakefulness to REM. This is an important feature in the case of patients with narcolepsy, who often transition to REM from being fully awake, without intermediate stages. In some embodiments the REM detector 26 combines information from the peripheral devices 6 relevant to muscle activity (which is absent in REM) with higher frequencies of brain bioelectrical activity characteristic of the REM stage to detect REM sleep.

The detector 22 may comprise one or more specific feature detectors 25-28, arranged to identify particular characteristics in the patient's bioelectrical activity. These specific feature detectors 25-28 are now described, though it is to be noted that other features associated with a state of reduced arousal and/or wakefulness may alternatively or additionally be used to identify reduced arousal/wakefulness.

The characteristic detected by the slow wave detector 25 is the presence of slow wave activity. Slow waves activity is described as oscillations in a range from 0.5 Hz to 4 Hz in EEG or deep brain recordings. Slow waves are a characteristic that can be associated with an early state of reduced arousal, herein referred to as the pre-sleep stage, while there is evidence that such frequency fluctuations in brain activity can reflect decrease in vigilance. Cortical slow wave activity has been shown to occur outside of sleep during low vigilance states for example in Huber et al., 2000, Vyazovskiy et al., 2011, Siclari and Tononi, 2017. One approach for the slow wave detector 25 is to map slow wave activity that occurs during transitions from an awake state to pathological sleep (e.g. daytime drowsiness, sleep attacks or excessive daytime sleepiness). It can also be used in disorders of consciousness where slow wave activity appears during the day.

The slow wave detector 25 may detect isolated waves of slow waves, and/or trains of slow waves. The slow wave detector 25 may detect slow wave activity using any suitable technique, as would be appreciated by those skilled in the art.

In one example, the slow wave detector 25 may calculate the power of the bioelectrical activity in one or more frequency bands from within the frequency range of slow waves from 0.5 Hz to 4 Hz. Suitable frequency bands may be 0.5 Hz to 1 Hz, 1 to 2 Hz, and 3 to 4 Hz. The slow wave detector 25 may then detect slow wave activity on the basis of the calculated power(s). Any suitable detection criteria may be used. In one, simple example, the detection criterion may be that the total power exceeds a threshold in a zero-crossing paradigm. Earlier examples of such methods have been used for slow wave detection in papers, such as that of Riedner et al. 2007. In another, more complex example, the detection criterion may compare the similarity of the profile of the powers across plural frequency bands with a characteristic profile of slow wave activity (such as sub-divisions of the 0.5 Hz to 4 Hz power band, or various locations—deep brain or cortical—of slow wave activity).

In another example, slow wave detection may be based on waveform amplitude. For instance, one can identify the relative average root mean square (RMS) in the one or more of the alpha, beta, and delta EEG frequency bands recorded from the monitoring transducer 3. The slow wave RMS can then be calculated as a moving average of the EEG activity within a time window, e.g. 10 seconds. Additional criteria can be implemented as desired, in accordance with the level of sleep depth that it is desired to inhibit. For instance, average slow wave RMS can be required to remain above a threshold level for a threshold period of time, for example above 10 μV for at least 75 s; or a criterion of slow waves per epoch/time window (e.g such as that must be six slow waves within a 30 s period) can be implemented—as has been described by Santostasi et al. 2016 for different purposes. If these conditions are met, then the slow wave detector 22 determines that there is slow wave activity sufficient to require stimulation.

It may be advantageous to consider regional slow wave activity profiles, and/or associated neuronal firing rate periods if the implanted electrode characteristics allow, as these can provide nuanced information as to early decreased arousal in a number of conditions, while there is evidence that some brain regions can still be active while others are not (Nir et al., 2011).

Slow waves (or any other characteristic detected by the detector 22) may be detected by comparison to activity observed in the patient. In other words, the detected characteristics may be patient-specific. For example, daytime peaks in slow wave frequency range can be recorded and correlated to behaviour in clinic indicative of reduced arousal/wakefulness. Feasibility data for such a detection strategy has been acquired, mapping intracranial activity of four different targets during three discrete levels of arousal (the transition from quiet wakefulness to a drowsy/less responsive state and then to sleep). A sample showcasing that the activity profile of a deep brain region visibly differs based on arousal level.

This is shown in FIG. 4a, which is a set of graphs of power spectral density (PSD) over time (in seconds) of slow wave activity in posterior insula for the three experimental conditions, namely quiet wakefulness which is the ‘night-time baseline’, drowsiness and sleep. There are scaling differences between the graphs.

Similarly, Table 1 shows a comparison of mean power of slow wave activity for several regions of interest within and across conditions (awake, drowsy, asleep). Slow wave activity was calculated based on a bipolar montage for equal volume of tissue between contacts.

TABLE 1
Mean SWA power (ROI per condition)

| Region of Interest | Night-time baseline | Drowsy | Sleep |
|---|---|---|---|
| Frontal EEG | 12.9624 | 18.9797 | 32.9290 |
| IFG | 16.4878 | 19.3359 | 83.9860 |
| Operculum | 1.0096 | 2.3076 | 12.2438 |
| Insula-middle | 37.0911 | 40.9744 | 146.2207 |
| Insula-posterior | 3.8798 | 4.1560 | 16.7736 |

In all four different deep brain areas, slow wave activity increases as the consciousness level decreases (from awake baseline to drowsiness to sleep).

This increase was statistically significant when transitioning from a quiet baseline to drowsiness. Pairwise t-tests with Bonferroni correction for the inferior frontal gyms (IFG) revealed a P=0.000 CI: [−4.1699-1.5262] while the middle insula had a P=0.0222 CI: [−6.7253-1.0412]. When the level of consciousness decreases further (drowsiness to sleep), increases are again statistically significant for all areas. (IFG: P=0.000 CI: [−75.3727-53.9274], Operculum: P=0.000 CI: [−11.5095-8.3628], Insula-middle: P=0.000 CI: [−119.1022-91.3905], Insula-posterior: P=0.000 CI: [−14.1321-11.1032]).

Similarly, this is shown in FIG. 4b, which is a plot of mean power spectrums in in the pedunculopontine nucleus of the brainstem which shows a decrease in beta and gamma activity as the level of arousal decreases. FIG. 4c shows how this modulation is most evident in the mapped space corresponding to the anatomical coordinates of the nucleus. These data therefore indicate that we can monitor the level of arousal from intracranial electrodes. This is also shown in FIG. 3a, where slow wave activity is visible in EEG and deep-brain/local field potential (LFP) measurements.

The slow wave activity detected by the slow wave detector 25 is a characteristic associated with state of reduced arousal that can precede sleep (drowsiness). Detecting slow waves is therefore particularly useful in the case where we want to avoid a transition from wakefulness to sleep. It can therefore trigger stimulation in an initial period of reduced wakefulness. This is illustrated in FIG. 3b, which shows how regional slow wave activity decreases when deep brain stimulation is delivered. The colour map illustrates slow wave power, with high slow wave power shown as light colours, as indicated in the bar in the figure. The left image shows slow wave power before application of stimulation. The right image shows slow wave power after application if stimulation. As can be seen, slow wave power is reduced after application of stimulation.

If this intervention succeeds then the patient will not reach other sleep stages (such as the formal sleep stages of non-REM Sleep Stage 1 and 2, (NREM-1 and NREM-2)). However, there are instances—such as in narcolepsy—where sleep comes in ‘attacks’ without any preceding drowsiness, classically with abrupt REM onset. For this reason, it is preferable to tailor the device, and the specific detector/s included in detector 22, to the needs of the patient and disease-state.

In the case of NREM-2 onset, the device may detect features characteristic of NREM-2 sleep. For example, sleep spindles and/or K complexes may be detected in the bioelectrical activity using the detecting mechanisms discussed below in relation to sleep fragility. As illustrated in FIG. 1, the sleep stage detector 22 may comprise a K-complex detector 27 and/or spindle detector 28 for detecting these features. The detector 22 may comprise only the K-complex detector 27 and/or spindle detector 28, for example in the case where only direct transitions to NREM-2 are relevant. Alternatively, the detector 22 may comprise both the slow wave detector 25, and one or both of the K-complex detector 27 and spindle detector 28. This combination may be useful where the stimulation in response to slow wave detection is not successful in modulation arousal/wakefulness, or where the slow wave detector 25 fails to detect a slow wave. Stimulation can then be triggered upon detection of a K-complex and/or spindle (and/or any other feature characteristic of NREM-2 sleep).

Further, any of the arousal/wakefulness detection mechanisms (pertaining to local field potentials as described here but also relevant to underlying neuronal activity, such as directly measured firing rates) may additionally take into account peripheral activity of the patient, which may be supplied to the detector 22 from peripheral monitoring devices 6 via interface unit 24. Peripheral activity may be cardiovascular activity (e.g. reduction in heart rate), musculoskeletal activity (e.g. change in muscle tone), and/or physical activity (e.g. as measured by an actigraph). The sleep stage detector 22 may make the determination that characteristics of reduced arousal/wakefulness are present based on the combination of characteristics detected in the bioelectric activity, and characteristics detected in the peripheral activity. For example, peripheral characteristics may be used to confirm a reduction in arousal/wakefulness identified in the bioelectrical activity.

In some examples, stimulation may be applied simply in response to detection of characteristics associated with reduced arousal/wakefulness. For example, stimulation may be applied upon detection of a slow wave, and until slow waves cease (or reduce in power/amplitude below a predetermined threshold). However, it may be beneficial to more carefully time the application of the stimulation to improve the chances of modulating wakefulness/arousal.

To this end, detection unit 20 in FIG. 1 comprises a sleep fragility detector 23 configured to detect an indication of sleep fragility/increased susceptibility to stimulation, in order to establish an optimal window for stimulation delivery so as to increase the level of arousal and/or induce wakefulness. Sleep fragility detector may identify indications of sleep fragility in the bioelectrical activity of the patient and/or the combination of bioelectrical and peripheral activity of the patient (the latter as provided to the sleep fragility detector 23 via interface unit 24). Indications of sleep fragility may be microarchitectural features of sleep or periodic features thereof or other established pattern of local field potential (LFP) or firing rate of the area of interest, associated with a higher probability of an electrical signal increasing the level of arousal. Identifying indications of sleep fragility may comprise identifying well-established features of susceptibility (such as microarousals and/or K-complexes), or by detecting more complex phenomena based on such features (such as the periodicity of spindles and periods of high neuronal firing—otherwise termed ‘ON’ states (Steriade et al. 2006)) or a combination of bioelectrical brain activity and peripheral metrics, creating patterns such as those identifiable through machine learning and/or modeling approaches. The sleep fragility detector may comprise one or more of the specific feature detectors 27-30 discussed below.

Perhaps the simplest way to time stimulation delivery during a period of sleep fragility would be to deliver it during or immediately after (e.g. within a predetermined period of) a micro-arousal. In FIG. 1, sleep fragility detector 23 comprises a microarousal detector 29 arranged to detect microarousals in the patient's bioelectrical activity. Microarousals are ultra-short periods of transient wakefulness, and are characterised by either fast, low-voltage high-frequency shifts or higher amplitude bursts of cortical activity (Halasz et al 2004). Optionally, peripheral data such as actigraphy data (e.g. motion/muscle activity) may be fed back to the device and evaluated together with brain activity. In such a (non-restrictive) example, the interface unit 24 would relay to sleep fragility detector 23 that a bodily movement had occurred, while the monitoring unit 21 would relay a signal containing frequency shifts and/or a high level of noise in the signal, over a predefined duration of time (such as, for instance, 5 seconds minimum). The concept of stimulus delivery during or after these short periods would be prolonging them into conscious wakefulness through constant, enhanced arousing input. However, a micro-arousal could occur after a variable period of pathological sleep, therefore a wider variety of features may be required for a fast and clinically effective paradigm.

There are a variety of micro-architectural features present during sleep that have been proposed to gate arousal. The term microarchitecture here implies that they are part of a distinct sleep stage, e.g. non-REM Sleep Stage 2, with sleep stages being the macro-architecture. The experimental paradigms that gave rise to such theories relied on sensory (mainly auditory) stimulation, but herein they are used to trigger electrical stimulation, for example deep brain stimulation or transcranial stimulation. In contrast to an exclusive use of microarousals, also (or alternatively) targeting specific fragility features of non-REM Sleep Stage 2, such as k-complexes and/or sleep spindles, may permits a relatively early intervention, where a transition to deep sleep is avoided.

The sleep fragility detector 23 in FIG. 1 comprises a k-complex detector 27 configured to detect k-complexes. A k-complex is a waveform that may be seen in a bioelectrical signal from the monitoring unit 21 and is a feature of non-REM Sleep Stage 2. K-complexes have been considered a salience decision point (decision to wake vs to remain asleep) at stimulus presentation, since asides from occurring spontaneously they can appear during a stimulus presentation in sleep (Jahnke et al 2012, Halasz et al 2016). Recently, it has been proposed that they are generated by important areas in the salience network (such as the dorsal anterior cingulate cortex (dACC) and are homologous to wakefulness error-related negativity (Ioannides et al. 2019). K-complexes take the form of a preceding positivity not always discernible by eye, followed by a large abrupt onset negativity in the EEG (>0.5 s and up to 0.3 mV), followed by a longer lasting positivity (Cote et al., 2000; Colrain, 2005). The arrows in FIG. 9 indicate the locations of an example K-complexes in EEG measurements from multiple electrodes.

The k-complex detector 27 may detect k-complexes in any suitable manner. One option would be to identify the relative average root mean square (RMS) of activity in the 0.5-4 Hz range during periods where sleep spindles are also present in the signal (so that it can K-complexes can be distinguished from isolated slow waves or slow wave activity in deep NREM-3 sleep). Further constraints may include that the onset amplitude is greater than a threshold for a predetermined period of time, for example up to 0.3 mV for >0.5 s, and then be followed by a longer change in polarity (signaling the larger onset positivity, since sometimes positive and negative can be flipped in different electrode montages). Alternatively or additionally, the k-complex detector 24 may comprise a waveform event detector that recognises events within bioelectrical signal from the monitoring unit 21 having a waveform that has a similarity, according to a similarity measure, with one or more stored waveforms that are representative of k-complexes encountered in practice. Another option is that the k-complex detector 24 is a machine learning detector that implements a machine learning algorithm that has been trained on training dataset of examples of k-complexes encountered in practice.

Stimulation may be triggered upon detection of such an event (K-complex) to take advantage of the gating period of arousal and therefore higher susceptibility of the network to respond to stimulation in the desired way (enhancing arousal and/or inducing wakefulness). Such a type of stimulation onset can be seen in FIG. 9 and its effects are also described in following sections of the disclosure, relevant to types of stimulation and their effects. This detection of the K-complex can be refined, for instance to deliver stimulation on the P200 or when there is a brief period of high-frequency (such as gamma) activity associated with the K-complex phase.

In FIG. 1, the K-complex detector 27 is shown as part of both the sleep stage detector 22 and the sleep fragility detector 23. This is because detection of K-complexes may be used for both purposes—both identifying that there is a reduced state of arousal and identifying that there is a sleep fragility to be exploited for optimising the stimulation timing. In alternative examples, the K-complex detector may be part of only the sleep stage detector 22 or the sleep fragility detector 23—in particular in cases where one of those components is omitted. Where both detectors 22, 23 are present, the detectors 22, 23 may share a common K-complex detector 27, for example which may feed the identification and/or properties of a detected K-complex into respective logic circuits for performing the respective functions of detectors 22, 23.

Both the sleep stage detector 22 and the sleep fragility detector 23 in FIG. 1 also comprise a spindle detector 28. The characteristics detected by the spindle detector 28 are sleep spindles. A sleep spindle (SS, sometimes referred to as sigma activity) is a waveform that may be seen in a bioelectrical signal from the monitoring unit 21. Examples of sleep spindles can be seen in FIG. 5, which shows EEG results of a function of time for multiple electrodes. Sleep spindles are most prominent in the regions highlighted by boxes in FIG. 1.

Sleep spindles are features of non-REM Sleep Stage 2 (NREM-2). Thus the sleep stage detector 22 may use the presence of sleep spindles to detect that the patient is in a sleep stage, and so may apply stimulation. Sleep spindles may be broadly defined as distinct waveforms in the 11-16 Hz frequency range. They are produced by cortico-thalamic loops and have been considered to stabilise sleep via inhibition of ascending arousing afferents (e.g. Wimmer et al. 2012), or cortical de-afferentation (Peyrache et al 2011). Sleep spindles are closely linked to k-complexes, typically preceding or following them during NREM-2 (Kokkinos and Kostopoulos, 2011). As such, the spindle detector 28 can be used by sleep stage detector 22 as an indicator that sleep has occurred (such as in the case of sleep attacks with abrupt transitions from wakefulness to NREM-2), as well as be linked to k-complex detector 27 to ensure accurate k-complex detection (during NREM-2 sleep).

The spindle detector 28 can also be used by/linked to sleep fragility detector 24 via the following mechanism. The occurrence of sleep spindles has been shown to have an intrinsic periodicity, called infraslow oscillation (Lecci et al. 2017). Infraslow oscillation may be defined as a bioelectrical signal with maximal spectral power in the frequency range 0.01-0.1 Hz, depending on the subject and on the cortical region to which the bioelectrical signal corresponds. By way of example, FIG. 6 shows a graph of normalised power spectral density (PSD) of such periodicity of sleep spindles against frequency of the periodic occurrence in an experimental study, wherein the spindle frequency was derived from a frequency range of 12-16 Hz. A maximal periodicity peak is noted close to the 0.05 Hz mark.

FIG. 10 shows further examples of PSD measurements of sleep spindles, measured pre-stimulation. In the top graph of FIG. 10, it is apparent that SS power is incrementally declining. In the lower graph of FIG. 10, it is apparent that SS power is incrementally increasing. When sigma power is on the decline (as part of the infraslow oscillation) an auditory stimulus has been shown to have a higher probability of waking a subject up compared to when sigma power was incrementing (Lecci et al 2017). Spindles are reportedly preserved in narcolepsy, despite altered distribution in Type 2 narcolepsy (Christensen et al. 2017), meaning that this infraslow oscillation may still be identifiable even in severe sleep disorders.

The spindle detector 28 may detect spindles in any suitable manner. A number of spindle detection algorithms are available in the art, for example either subdividing spindles in slow and/or fast spindles or looking at different amplitude and topographical features. One such being in accordance with methods described by Lecci et al. 2017, incorporated herein by reference, —where a method for calculation of the infraslow periodicity is also described. We successfully implemented a method calculating the period in the normalized power in band for fast (13-16 Hz) and slow (11-13 Hz) spindles in a similar manner to that described by Lecci et al. 2017 in the detection part of diagrams illustrating this disclosure. Stimulation triggered during the offset (decline) of the infraslow spindle oscillation may be more successful in inducing arousal/wakefulness and therefore enhance the efficacy of the stimulation.

As with the K-complex detector 27, the spindle detector 28 may be shared by both detectors 22, 23. The K-complex may, in alternative examples, be part of only one of the sleep stage detector 22 and the sleep fragility detector 23, in particular where one of those components is omitted.

Alternatively or additionally, sleep fragility detector 23 may comprise a pattern detector 30 arranged to identify patterns, in the patient's bioelectrical activity (which could include neuronal firing rates in the case of implanted laminar microelectrodes) and/or in the combination of bioelectrical and peripheral activity, corresponding to fragility/susceptibility. Such patterns need not be directly dependent on detection of micro-architectural sleep features, such as those described above. In particular, pattern detector 30 may comprise a machine learning algorithm (or other algorithm) trained to identify signals indicative of sleep fragility. The algorithm may be trained based on prior analysis of patient-specific data of network bioelectrical activity and/or peripheral activity (such as but not limited to changes in heart rate and activity levels) algorithm. Recognition of a pattern may be used to establish optimal windows for the stimulation. Such a detection algorithm may operate online (processing ongoing activity) and advising stimulation onset in cooperation with other detectors (such as detection unit 22) to trigger the onset and offset of stimulation.

The stimulation generator 31 generates and terminates stimulation signals in response to the sleep stage detector 22 and/or the sleep fragility detector 23 (and/or any of the detectors 25-30 encompassed therein, with all their relevant connections and optionally interface 24 input). The generated stimulation signals are supplied via the wire 5 to the stimulation transducers 4. This causes the stimulation transducers 4 to apply electromagnetic stimulation to the target region, either directly in the case that the stimulation transducers 4 are implanted (where the stimulation may particularly by in the form of electrical stimulation) or indirectly in the case that the stimulation transducers 4 are external (where the electromagnetic stimulation may be in the form of electrical or magnetic or both electrical and magnetic stimulation).

The stimulation signals are selected to arouse the patient when the stimulation is applied. In general terms, these signals may be selected based on the following principles. Early experiments revealed that brain activation is characterised by synchronisation and enhancement of fast cortical rhythms (30-40 Hz) (Steriade et al 1996). This is practically reflected in gold-standard sleep scoring criteria, where wakefulness and light sleep are described by predominance of higher frequencies, such as the gamma band having a frequency range 25 Hz-140 Hz, the beta band having a frequency range 12.5 Hz-30 Hz and the alpha rhythm having a frequency range 8 Hz to 12 Hz (Berry et al. 2015). High-frequency cortical activity has been shown to be driven by basal forebrain projections (parvalbumin interneurons) (Kim et al 2015). It has been proposed that in the RAS, gamma activity may stabilise brain area coherence relevant to arousal (Urbano et al. 2012, Garcia-Rill et al 2013). There is also additional evidence that high-frequency firing in hypothalamic neurons triggers the vesicular release of orexin (Adamantidis et al 2007, Yamanaka et al. 2010).

The value of this information is two-fold. Firstly, in order to engage the brain in wakefulness-related activity the stimulation device 1 should mimic these known intrinsic patterns to achieve a naturalistic, physiological effect. In particular, the simulation signals may comprise one or more features (e.g. frequency) representative of an endogenous activity pattern associated with arousal/wakefulness, such as alpha, low gamma, or high gamma activity. Secondly, brain activity compatible with wakefulness could serve as an objective metric for the evaluation of the efficacy of such a device.

The stimulation generator 31 may comprise plural independent current sources that can drive patterns of stimulation over the desired range. Cycling allows for bursts of activity and sub-Hz functionality. The current output levels and other parameters of the current sources align with the desired stimulation signals. Where plural stimulation transducers 4 are present, the stimulation signals may be provided over plural channels to different sets of electrodes. Such channels are current-controlled and charge-balanced.

Examples of suitable stimulation signals are as follows.

The stimulation signal may comprise at least one stimulation pulse repeating in a cycle, for example a pair of stimulation pulses of opposite polarity. FIG. 7 illustrates an example of such a stimulation signal 71 as a graph of voltage against time (with arbitrary units). The stimulation signal 71 comprises a pair of stimulation pulses 72, 73 of opposite polarity repeating in a cycle. The opposite polarity of the stimulation pulses 72, 73 helps to prevent charge build-up in the target region. The stimulation signal 71 has a stimulation frequency Fₛ=Tₛ/1 where Tₛ is the time period of the stimulation signal 71, i.e. the length in time of the repeating cycle of the stimulation signal 31. As shown in FIG. 7, the time period Tₛ is the time between equivalent points on consecutive stimulation pulses of the stimulation signal 71. The stimulation pulses 72, 73 have respective widths W1, W2 which may be different. Similarly, the stimulation pulses 72, 73 have amplitudes which may be different. FIG. 7 illustrates an example where there is a gap of length Wi between the stimulation pulses 32, 33, but that is not essential.

A stimulation signal comprising pulses is straightforward to generate, but such a waveform is not essential and in general the stimulation signal may have any waveform.

In some embodiments, the stimulation signals may have a stimulation frequency in a high frequency range from a lower limit of 8 Hz, more preferably 40 Hz to an upper limit of 150 Hz, or preferably 120 Hz. It is possible that the optimal stimulation frequency will differ according to target nucleus, stage/type of sleep disorder or individual patient. However, it is anticipated that such variations will nonetheless within this frequency range. In addition, it will be feasible to determine these individual parameters post-implantation during an inpatient stay and program the device to deliver the optimal stimulus subsequently. Pulse width and amplitude may also be configured in clinic sessions and optimized based on device parameters and patient tolerance.

Studies have been performed demonstrating that electrical stimulation of a target region in the ascending arousal system in humans in a frequency within the prespecified range can reproducibly reduce activity associated with greater sleep depth and increase power in frequencies associated with increased arousal and alertness.

In all the studies, the target region was the same (pedunculopontine nucleus or PPN), the stimulation signal 31 shown in FIG. 7 was used, and the pulse width and amplitude of the stimulation pulses 72, 73 were also kept constant in all experiments. The pulse width and amplitude of stimulation pulses were tailored to levels that would not cause any sensory disturbances to the patient (patients did not have any sensations—such as buzzing/tingles of body parts) that could inform them of the fact that the stimulator was on and/or disturb their sleep because of their effects.

In one such series of experiments, bilateral stimulation (using two electrodes to stimulate nuclei of interest on both sides, as would happen clinically) was applied during 12 trials in three patients. The stimulation was delivered in response to K complex detection. Both pre-stimulation periods were comparable in terms of power density of slow wave activity. The stimulation frequency used was in the gamma frequency range (100 Hz), with amplitude and pulse width remaining constant per patient at a subthreshold level. The results are shown in FIG. 8a which is a set of four graphs and a table. The graphs illustrate comparisons for the mean PSD in four frequency bands (slow wave activity—SWA, gamma, theta, beta) during an equal pre- and post-stimulation period (at least 3 epochs of 30 s, total length dependent on post-stim movement artefact). The table illustrate the results numerically. In the table, H is the level of significance (1=statistically significant, 0=non-significant); P is the p-value; CI is the confidence interval; PRE—is the mean PSD pre-stimulation; and POST—is the mean PSD post-stimulation. The level of significance illustrated survived Bonferroni correction for multiple comparisons, showing that high frequency stimulation effectively caused transition to lighter sleep or sustained wakefulness during 12 trials in three patients.

In a second series of experiments, short bursts (≤5 mins) of unilateral stimulation at either 40 Hz or 100 Hz were applied to study the efficacy in reducing SWA and increasing rhythms associated with wakefulness/lighter sleep. The results are shown in FIG. 8b which is three pairs of figures and three accompanying tables showing a comparison (paired-sample t-tests) between mean power spectral densities during an equal pre- and post-stimulation period (at least 3 epochs of 30 s, total length dependent on post-stim movement artefact). In FIG. 8b: H is the level of significance (1=statistically significant, 0=non-significant); P is the p-value; CI is the confidence interval; mean_pre is the mean power pre-stimulation; mean_post is the mean power post-stimulation; mean_40 is the mean power pre-stimulation at 40 Hz; and mean_100 is the mean power pre-stimulation at 100 Hz. Results are illustrated for the following frequency bands: alpha, gamma, and SWA. There were also statistically significant increases in beta (P=0.0017 and P=0.027 for stimulation at 100 Hz and 40 Hz respectively). The results show that both protocols induce higher (wake-related) cortical activity, that is, but the higher frequency (100 Hz) was more effective at reducing slow wave activity and shifting brain activity to ‘wakefulness’ frequencies.

In additional experiments, unilateral stimulation was delivered during deep sleep (stage 3 non-rem sleep NREMS-3). That is, stimulation was delivered without any association with features of fragility but in response to slow wave activity (SWA) associated with a decrease in level of arousal. Although neither unilateral 100 Hz nor unilateral 40 Hz stimulation decreased slow wave power to a statistically significant degree, they were efficient in inducing faster rhythms associated with lighter sleep and increased arousal levels. In particular, both frequencies increased alpha power (P=0.0000 CI [−0.0574-0.0203] for 40 Hz, P=0.0026 CI [−0.0621-0.0133] for 100 Hz). Also, unilateral stimulation at 100 Hz successfully increased gamma power (P=0.0000 CI [−0.0004 0.0004].

FIG. 8c shows the results of an additional efficacy control, in which all trials across four patients were compared to selected ‘sham’ epochs (where no stimulation was delivered, but the pre-stimulation neurophysiological activity was comparable to trials where stimulation would have been delivered. A two way Anova (factors g1=stim, SHAM, g2=pre, post) was performed for transformed PSDs (SWA λ=−0.073 and G λ=−0.102). The full model, i.e. interactions included with Bonferroni correction, revealed that there was a sustained significant decrease in cortical SWA (0.5-4 Hz) from pre-stimulation (p=0.0000, CI [0.2675 0.3537) and a significant increase in gamma (30-60 Hz) activity (p=0.0000, CI [−0.4517-0.3256], even after comparison with sham trials.

The above results demonstrate efficacy in patients showing slow wave activity as can occur in various conditions associated with decreased arousal. Such stimulation signals can therefore be used in a closed-loop protocol that triggers stimulation in response to the output of the slow wave detector 25 to disrupt these transitions therefore restoring wakefulness. Given that this restoration may take more than one stimulation pulse to achieve, the stimulation device can be programmed to ramp up pulse width and amplitude if a wake rhythm has not been restored. Such a ramp may occur up to a threshold set by a clinician based on individual tolerance and clinical safety. Should the condition where the stimulation device 1 is used be linked to sleep, and slow wave activity (such as in NREM-3) give way to a lighter stage of NREM-2, then additional detectors (such as the k-complex detector 27 or the sleep fragility detector 23) can then be triggered and wakefulness successfully restored.

Such a stimulation pattern can also be informed by secondary variables in addition to deep brain activity. For example, the device can also incorporate activity profiles by linking in with an actiwatch (portable activity device) to estimate what the subject is doing when drowsiness ensues (stationary vs moving/driving), enabling the option of an automated alert to be recorded or sent to the healthcare team in high risk situations. This is an additional use for interface unit 24. There could also be an option for the patient to self-activate the stimulation system in such cases and deliver stimulation on demand (e.g. when the patient becomes aware of an impending ‘sleep attack’).

The device description and results disclosed allow for multiple different stimulation delivery strategies all allowing for enhancement of arousal and modulating wakefulness to various degrees. We will proceed to describe a non-limiting number of such approaches/stimulation strategies.

One approach referred to herein as a global approach is to generate the stimulation signals on detection of features that are hallmarks of decreased arousal (such as slow wave activity detected by slow wave detector 25). Such features may be shared in a variety of disorders (such as but not limited to minimally conscious state, onset of drowsiness in hypersomnia in neurodegeneration and certain sleep stages in sleep attacks of narcolepsy). The stimulation signal may be applied until the conditions triggering stimulation cease to be detected. In the case of slow waves, the stimulation generator 31 may generate the stimulation signals in response to multiple occurrences of slow waves, or a previously defined threshold, as discussed above in relation to slow wave detector 25. A stimulation signal (comprising repeating pulses) may be applied for an extended period of time, until a level of increased arousal is detected. In this example this could happen if slow wave detector 25 stops detecting slow wave activity (or detects SWA below a certain threshold) and therefore stops triggering the stimulation generator 31, stopping the stimulation. This could also happen via the interface unit 24 detecting increases in peripheral activity, for example movements, optionally accompanied by an increase in heart rate, signaling arousal. Then the interface unit 24 would communicate this to the detector 22 and together these inputs would stop the triggering of stimulation generator 31. This is an example of an adaptive stimulation loop using the system. When such a global approach is used, the device 1 may not comprise a sleep fragility detector 23 (and shared detectors such as K-complex detector 27 and spindle detector 28, if present, may be part of sleep stage detector 22 only).

Another approach (that could optionally be combined with/incorporated in stages of the previous approach) is to synchronise the generation of the stimulation signals with the detection of events or patterns relevant to sleep fragility (or signaling an increased susceptibility of the network to respond to stimulation and enhance arousal). As described previously such events may for example be microarousals, k-complexes and/or sleep spindle periodicity (detected by the microarousal detector 29, the k-complex detector 27 and the spindle detector 28, as part of sleep fragility detector 23). As also described previously, where the system/implanted electrode capacity permits, this could relate to other patterns such as those of neuronal firing activity. Accordingly, the maximal efficacy of stimulation may be achieved by delivering the stimulation at such times. This achieves the desirable clinical outcome more effectively and faster, but also conserves of power. Power conservation is important as the stimulation device 1 is typically powered by a battery 2 so achieves cost reduction through less need for battery replacement surgeries.

In our experimental series disclosed as part of this draft, detection of k-complexes triggered the stimulation within a second from feature onset, and five minutes of stimulation at a tailored frequency were sufficient to enhance arousal. The duration of the pulses delivered in such a shorter loop (again, responsive closed-loop stimulation) would depend of fragility feature detected—in a sense, how close to the higher desired arousal level the patient is. In the same vein as described in the previous method, the stimulation generator 31 may stop delivering pulses either when the detector 22 stops detecting features associated with decreased arousal level and/or the interface 24 detects peripheral activity; or the stimulation generator 31 may change to a different stimulation strategy when a fragility feature is no longer detected by susceptibility detector 24.

An automated detection approach (based on either EEG or ideally intracranial activity) could deliver pulses with additional levels of precision (such as always delivering the pulse during one slope of the waveform, according to optimal network response). FIG. 9 shows a set of bioelectrical signals from EEG monitoring transducers during the delivery of electrical stimulation to the PPN in response to a k-complex. Stimulus delivery indicated by arrows also pointing towards the two different phases of the waveform. Our manual approach meant that we could not be phase-specific with regards to stimulus delivery (if the stimulation always happened during the positive vs the negative phase of the waveform—which as specified above can have differences in its likelihood with associations with faster rhythms such as small bursts of gamma activity).

The features used to trigger the stimulation may be individual to a specific pathology or a specific patient. In this case, characteristics of the features representing the transition from wakefulness into sleep may be identified during baseline monitoring of different patients having a specific pathology or of a specific patient. Then, the identified characteristics of the features may be used to trigger the stimulation generator 31.

Sleep stages with a higher probability of transition to wakefulness could also be identified using data-driven methods (such as described in Stevner at al. 2019). A machine-learning approach would mean that the device is constantly optimised during its use, since every additional night will inform the training algorithm. Any concerns with regards to risk could be mitigated by allowing the device to close the loop and deliver targeted stimulation based on such an algorithm only if transition probability of detected states exceeds a threshold of accuracy, that has been set and reviewed by the patient's clinical team. These states could be tailored to each patient and thus take parameters such as age, disease type and stage into consideration. This is also particularly important since there is evidence of disease-specific dysfunction in state transitions in disorders such as narcolepsy (Ferri et al. 2016).

It is possible to apply one of these global and individualised approaches alone, or both in combination.

The multi-layered approach implemented in the stimulation device 1 combines combining interventions during universal and unique features to ensure maximal efficacy. However, to ensure appropriate risk mitigation (especially during periods such as high activity levels) the stimulation device 1 could communicate directly with the clinical team by sending an alert and status update whenever stimulation is triggered. In addition, data acquired could be stored in a secure cloud so that they could be accessed and evaluated when desired, to ensure optimal treatment safety and efficacy.

That said, it is not essential for the stimulation device 1 to include each of the slow wave detector 25, the microarousal detector 29, the REM probabilistic detector 28, the k-complex detector 27, spindle detector 28, and the pattern detector 30. In general any one or more of these units may be included in the device.

For instance in some embodiments, the REM probabilistic detector 28 may be omitted, so that the stimulation device 1 only triggers stimulation on the basis of features of susceptibility (detector 23) and slow wave activity (detector 25) with the use of the interface unit 24. This approach may be suitable, for example, when the clinician wants to enhance arousal level and target pathological slow wave activity in minimally conscious state. This two-level strategy system could therefore be configured in a variety of individualised combinations to best facilitate treatment.

Further, the presence of the K-complex and/or spindles as detected by the K-complex detector 27 and/or spindle detector 28 acting as part of the sleep fragility detector 23 may in itself identify that the patient is in a reduced stage of arousal/wakefulness, as well as providing an optimized timing for the stimulation. Thus there may be no need for a dedicated step of identifying characteristics of a sleep stage, and so sleep stage detector 22 may be omitted.

FIG. 11 illustrates a method of generating stimulation signals for modulating the arousal and/or wakefulness level of a patient. For example, the method may be implemented using a stimulation device such as device 1.

The method comprises, at step 1101, monitoring bioelectrical activity of the nervous system of the patient. This may comprise receiving a signal from a monitoring transducer, as described above.

At step 1102, characteristics of the monitored bioelectrical activity associated with a state of reduced arousal or wakefulness are detected. This may comprise any of the detection techniques discussed above in relation to detection unit 20, and the components thereof.

At step 1103, in response to detecting said characteristics of the monitored bioelectrical activity associated with a state of reduced wakefulness, stimulation signals are generated for supply to transducers arranged to apply stimulation to a neural network of a patient associated with arousal, the stimulation signals being selected to arouse the patient when the stimulation is applied. The simulation signals may be generated in accordance with any of the stimulation schemes discussed above.

The method may further comprise any method discussed above as a function of the components of device 1, or any other example of a stimulation device discussed above.

As will be appreciated by the skilled person, the method may be performed by a processor arranged to execute instructions (e.g. stored in a memory), which, when executed, cause the processor to perform the method. In particular, the processor may be arranged to receive a signal from monitoring transducers, and to output a signal for controlling the stimulation transducers. Any of the functions of the detection unit 20, and components thereof (and optionally also the stimulation generator 30), may be implemented using such a processor.

### REFERENCES

Adamantidis A R, Zhang F, Aravanis A M, Deisseroth K & de Lecea L Neural substrates of awakening probed with optogenetic control of hypocretin neurons. Nature 450, 420-424 (2007)
Berry R B, Gamaldo C E, Harding S M, et al. AASM Scoring Manual Version 2.2 Updates: New Chapters for Scoring Infant Sleep Staging and Home Sleep Apnea Testing. J Clin Sleep Med. 2015; 11(11): 1253-1254. Published 2015 Nov. 15. doi: 10.5664/jcsm.5176
Bhat S, Chokroverty S. Hypersomnia in Neurodegenerative Diseases. Sleep Med Clin. 2017; 12(3):443-460. doi: 10. 1016/j.jsmc.2017.03.017
Carter M E, Yizhar O, Chikahisa S, Nguyen H, Adamantidis A, Nishino S, Deisseroth K, de Lecea L Tuning arousal with optogenetic modulation of locus coeruleus neurons. Nat Neurosci. 2010 Dec; 13(12): 1526-33
Christensen J A E, Nikolic M, Hvidtfelt M, Kornum B R, Jennum P. Sleep spindle density in narcolepsy. Sleep Med. 2017 June; 34:40-49. doi: 10. 1016/j.sleep.2017.02.022. Epub 2017 Mar. 18. PMID: 28522097.
Colrain, I. M (2005). “The K-complex: a 7-decade history.” Sleep 28, 255-273. doi: 10.1093/sleep/28.2.255
Cote K A, de Lugt D R, Langley S D, Campbell K B. Scalp topography of the auditory evoked K-complex in stage 2 and slow wave sleep. J Sleep Res. 1999; 8(4):263-272. doi: 10. 1046/j. 1365-2869. 1999.00164.x
Cruse D, Thibaut A, Demertzi A, et al. Actigraphy assessments of circadian sleep-wake cycles in the Vegetative and Minimally Conscious States [published correction appears in BMC Med. 2018 Aug. 11; 16(1): 134]. BMC Med. 2013; 11: 18. Published 2013 Jan. 24. doi: 10. 1186/1741-7015-11-18
Dauvilliers Y, Lopez R, Ohayon M, Bayard S. Hypersomnia and depressive symptoms: methodological and clinical aspects. BMC Med. 2013; 11:78. Published 2013 Mar. 21. doi: 10.1186/1741-7015-11-78
Ferri R, Pizza F, Vandi S, Iloti M, Plazzi G. Decreased sleep stage transition pattern complexity in narcolepsy type 1. Clin Neurophysiol. 2016 August; 127(8):2812-2819. doi: 10.1016/j.clinph.2016.05.364. Epub 2016 Jun. 8. PMID: 27417057.
Garcia-Rill E, Kezunovic N, Hyde J, Simon C, Beck P, Urbano F J. Coherence and frequency in the reticular activating system (RAS). Sleep Med Rev. 2013; 17(3):227-238. doi:10.1016/j.smrv.2012.06.00
Halász, P. (2016). “The K-complex as a special reactive sleep slow wave—a theoretical update.” Sleep Med. Rev. 29, 34-40. doi: 10.1016/j.smrv.2015.09.004
Halász P, Terzano M, Parrino L, Bodizs R. The nature of arousal in sleep. J Sleep Res. 2004; 13(1): 1-23. doi:10.1111/j.1365-2869.2004.00388.x
Huber R, Deboer T, Tobler I. Topography of EEG dynamics after sleep deprivation in mice. J Neurophysiol. 2000 October; 84(4): 1888-93. doi: 10.1152/jn.2000.84.4.1888. PMID: 11024081.
Ioannides A A, Liu L, Kostopoulos G K. The Emergence of Spindles and K-Complexes and the Role of the Dorsal Caudal Part of the Anterior Cingulate as the Generator of K-Complexes. Front Neurosci. 2019; 13:814. Published 2019 Aug. 7. doi:10.3389/fnins.2019.00814
Jahnke, K et al. (2012). “To wake or not to wake? The two-sided nature of the human K-complex.” Neuroimage 59, 1631-1638. doi: 10.1016/j.neuroimage.2011. 09.013
Kim T, Thankachan S, McKenna J T, et al. Cortically projecting basal forebrain parvalbumin neurons regulate cortical gamma band oscillations [published correction appears in Proc Natl Acad Sci USA. 2015 May 26; 112(21): E2848]. Proc Natl Acad Sci USA. 2015; 112(11):3535-3540. doi:10.1073/pnas.1413625112
Kokkinos, V., and Kostopoulos, G. K (2011). “Human non-rapid eye movement stage II sleep spindles are blocked upon spontaneous K-complex coincidence and resume as higher frequency spindles afterwards.” J Sleep Res. 20, 57-72. doi: 10.1111/j.1365-2869.2010.00830.x
Lecci S, Fernandez L M, Weber F D, Cardis R, Chatton J Y, Born J, Lûthi A. Coordinated infraslow neural and cardiac oscillations mark fragility and offline periods in mammalian sleep. Sci Adv. 2017 Feb. 8; 3(2): e1602026. doi: 10.1126/sciadv.1602026. Erratum in: Sci Adv. 2017 Sep. 29; 3(9): eaaq0565. PMID: 28246641; PMCID: PMC5298853.
Nir Y, Staba R I, Andrillon T, Vyazovskiy V V, Cirelli C, Fried I, Tononi G. Regional slow waves and spindles in human sleep. Neuron. 2011 Apr. 14; 70(1): 153-69. doi: 10.1016/j.neuron.2011.02.043. PMID: 21482364; PMCID: PMC3108825.
Peyrache A, Battaglia F P, Destexhe A. Inhibition recruitment in prefrontal cortex during sleep spindles and gating of hippocampal inputs. Proc Natl Acad Sci USA. 2011; 108(41):17207-17212. doi:10.1073/pnas.1103612108
Riedner B A, Vyazovskiy V V, Huber R, et al. Sleep homeostasis and cortical synchronization: III. A high-density EEG study of sleep slow waves in humans. Sleep. 2007; 30(12): 1643-1657. doi:10.1093/sleep/30.12.1643
Saini P, Rye D B. Hypersomnia: Evaluation, Treatment, and Social and Economic Aspects. Sleep Med Clin. 2017; 12(1):47-60. doi:10.1016/j.jsmc.2016.10.013
Samn S. and Perelli L. (1982). ‘Estimating aircrew fatigue: A technique with application to airlift operations (SAM-TR-82-21)’. Brooks Air Force Base, TX: U.S. Air Force.
Santostasi, G., Malkani, R., Riedner, B., Bellesi, M, Tononi, G., Paller, K A., et al. (2016). Phase-locked loop for precisely timed acoustic stimulation during sleep. J. Neurosci. Methods 259, 101-114. doi: 10.1016/j.jneumeth.2015.11.007
Saper C B, Scammell T E, Lu J. Hypothalamic regulation of sleep and circadian rhythms. Nature. 2005; 437 (7063): 1257-1263. doi: 10. 1038/nature04284
Schoch S F, Werth E, Poryazova R, Scammell T E, Baumann C R, Imbach L L. Dysregulation of Sleep Behavioral States in Narcolepsy. Sleep. 2017; 40(12): 10.1093/sleep/zsx170. doi: 10. 1093/sleep/zsx170
Sharma V D, Sengupta S, Chitnis S, Amara A W. ‘Deep Brain Stimulation and Sleep-Wake Disturbances in Parkinson Disease: A Review’, Frontiers in neurology, 2018; 9: 697-97
Siclari F, Tononi G. Local aspects of sleep and wakefulness. Curr Opin Neurobiol. 2017 Jun; 44:222-227. doi: 10.1016/j.conb.2017.05.008. Epub 2017 May 30. PMID: 28575720; PMCID: PMC6445546.
Sitt J D, King J R, El Karoui I, et al. Large scale screening of neural signatures of consciousness in patients in a vegetative or minimally conscious state. Brain. 2014; 137(Pt 8):2258-2270. doi: 10. 1093/brain/aw u 141
Steriade M, Amzica F, Contreras D. Synchronization of fast (30-40 Hz) spontaneous cortical rhythms during brain activation. J Neurosci. 1996; 16(1):392-417. doi: 10.1523/JNEUROSCI. 16-01-00392.1996
Steriade M Grouping of brain rhythms in corticothalamic systems. Neuroscience. 2006; 137:1087-1106
Stevner A B A, Vidaurre D, Cabral J, et al. Discovery of key whole-brain transitions and dynamics during human wakefulness and non-REM sleep. Nat Commun. 2019; 10(1): 1035. Published 2019 Mar. 4. doi: 10.1038/s41467-019-08934-3
Urbano F J, Kezunovic N, Hyde J, Simon C, Beck P, Garcia-Rill E. Gamma band activity in the reticular activating system. Front Neurol. 2012; 3:6. Published 2012 Jan. 31. doi: 10.3389/fneur.2012.00006
Vyazovskiy V V, Olcese U, Hanlon E C, Nir Y, Cirelli C, Tononi G. Local sleep in awake rats. Nature. 2011 Apr. 28; 472(7344):443-7. doi: 10.1038/nature10009. PMID: 21525926; PMCID: PMC3085007.
von Economo, K (May 10, 1917). “Die Encephalitis lethargica”. Wiener klinische Wochenschrift [Vienna clinical weekly] (in German). Leipzig and Vienna: Franz Deuticke 1918. 30: 581-585.
Wimmer R D, Astori S, Bond C T, et al. Sustaining sleep spindles through enhanced SK2-channel activity consolidates sleep and elevates arousal threshold. J Neurosci. 2012; 32(40): 13917-13928. doi:10.1523/JNEUROSCI.2313-12.2012
Yamanaka A, Tabuchi S, Tsunematsu T, Fukazawa Y & Tominaga M Orexin directly excites orexin neurons through orexin 2 receptor. J. Neurosci 30, 12642-12652 (2010).

1. A method of generating stimulation signals for modulating the arousal and/or wakefulness of a patient, the method comprising:
monitoring bioelectrical activity of the nervous system of the patient;
detecting characteristics of the monitored bioelectrical activity associated with a state of reduced arousal and/or wakefulness;
in response to detecting said characteristics of the monitored bioelectrical activity associated with a state of reduced arousal and/or wakefulness, generating stimulation signals for supply to transducers arranged to apply electromagnetic stimulation to a neural network of a patient associated with arousal, the stimulation signals being selected to arouse the patient when the stimulation is applied.

2. A method according to claim 1, wherein the step of monitoring bioelectrical activity comprises monitoring bioelectrical activity by electroencephalography.

3. A method according to claim 1, wherein the step of monitoring bioelectrical activity comprises monitoring bioelectrical signals derived from transducers implanted in the neural network of a patient associated with arousal, or from transducers placed on the scalp of a patient, or from transducers implanted epidurally or subdurally into the patient.

4. A method according to claim 1, wherein the transducers comprise electrodes and the electromagnetic stimulation is electrical stimulation.

5. A method according to claim 1, wherein the transducers comprise coils.

6. A method according to claim 1, wherein the characteristics are indicative of a stage of sleep.

7. A method according to claim 6, wherein the detected characteristics comprise slow wave activity.

8. A method according to claim 6, wherein the detected characteristics comprise features of non-rapid eye movement Sleep Stage 2, wherein optionally the features comprise microarousals and/or the features comprise k-complexes and/or the features comprise sleep spindles.

9. A method according to claim 6, wherein the detected characteristics comprise features of the rapid eye movement sleep stage.

10. A method according to claim 1, wherein the detected characteristics are features of sleep fragility in the patient, and optionally the stimulation signals are synchronized with the indication of sleep fragility.

11. A method according to claim 10, wherein detecting an indication of sleep fragility comprises detecting at least one of: a microarousal, a K-complex, and a sleep-spindle.

12. A method according to claim 10, wherein detecting an indication of sleep fragility comprises applying a machine learning algorithm trained to identify signals indicative of sleep fragility.

13. A method according to claim 1, further comprising:
monitoring peripheral activity of the patient; and
detecting characteristics of the monitored peripheral activity associated with a reduced state of wakefulness and/or arousal,
wherein the stimulation signal is generated in response to detecting the characteristics of the monitored bioelectrical activity and the characteristics of the monitored peripheral activity.

14. A method according to claim 13, wherein the peripheral activity comprises at least one of: cardiovascular activity, musculoskeletal activity, or physical activity.

15. A method according to claim 1, wherein the detected characteristics are patient-specific.

16. A method according to claim 1, wherein the neural network of the patient associated with arousal is a part of the ascending arousal system, wherein optionally the part of the ascending arousal system is the pedunculopontine nucleus or laterodorsal tegmentum.

17. A method according to claim 1, wherein the stimulation signals have a stimulation frequency of at least 10 Hz and/or at most 150 Hz.

18. A method according to claim 1, wherein the stimulation signal comprises at least one stimulation pulse repeating in a cycle, optionally comprising a pair of stimulation pulses of opposite polarity.

19. A method according to claim 1, wherein the stimulation signals comprise one or more features representative of an endogenous activity pattern associated with arousal and/or wakefulness.

20. A method according to claim 19, wherein the stimulation signals comprise one or more features representative of one of: alpha, low gamma, or high gamma activity.

21. A method according to claim 1, further comprising supplying the stimulation signals to the transducers.

22. A stimulation device for generating stimulation signals for modulating the wakefulness and/or arousal level of a patient, the stimulation device comprising:
a detection unit arranged to monitor bioelectrical activity of the nervous system of the patient and to detect characteristics of the monitored bioelectrical activity associated with a state of reduced arousal and/or wakefulness; and
a stimulation generator arranged, responsive to detecting said characteristics of the monitored bioelectrical activity associated with a state of reduced wakefulness, to generate stimulation signals for supply to transducers arranged to apply electromagnetic stimulation to a neural network of a patient associated with arousal, the stimulation signals being selected to arouse the patient when the electrical stimulation is applied.

23. A stimulation device according to claim 22, wherein the detection unit comprises a sleep stage detector arranged to detect characteristics indicative of a state of sleep of the patient in the monitored bioelectrical activity.

24. A stimulation device according to claim 22, wherein
the detection unit comprises a sleep fragility detector arranged to detect an indication of sleep fragility in the patient in the monitored bioelectrical activity, and
the stimulation generator is arranged to synchronize the stimulation signal with the detected indication of sleep fragility.

25. A stimulation device according to claim 22, further comprising the transducers and/or an interface unit configured to receive signals indicative of peripheral activity of the patient.
